(19) United States
(12) Patent Application Publication
(10) Pub. No.: US 2011/0137419 A1
(43) Pub. Date: Jun. 9, 2011

(54) BIOCOMPATIBLE TANTALUM FIBER SCAFFOLDING FOR BONE AND SOFT TISSUE PROSTHESIS

(76) Inventor: James Wong, Shrewsbury, MA (US)

(21) Appl. No.: 12/961,209

(22) Filed: Dec. 6, 2010

Related U.S. Application Data
(60) Provisional application No. 61/266,911, filed on Dec. 4, 2009.
(60) Provisional application No. 61/295,063, filed on Jan. 14, 2010.
(60) Provisional application No. 61/314,878, filed on Mar. 17, 2010.

(51) Int. Cl. A61F 2/28; A61F 2/02; A61B 17/04

(52) U.S. Cl. 623/16.11; 623/23.72; 606/228

(57) ABSTRACT

A tissue scaffolding agent for repair and regeneration of bone and soft cell tissue.

### CROSS REFERENCE TO RELATED APPLICATIONS

This application claims priority from U.S. Provisional Application Serial No. 61/266,911, filed Dec. 4, 2009, U.S. Provisional Application Ser. No. 61/295,063, filed Jan. 14, 2010 and U.S. Provisional Application Ser. No. 61/314,878 filed Mar. 17, 2010, the contents of which applications are incorporated herein, in their entirety, by reference.

### FIELD OF THE INVENTION

The present invention relates to the use of extremely fine tantalum fibers as a scaffolding agent for the repair and regeneration of both bone and soft cell tissue. These include solid body parts bone replacement implants such as knee, hip joints, as well as for soft tissue types such as nerve, tendons, cartilage, including body organ parts and will be described in connection with such utility, although other utilities are contemplated

### BACKGROUND ART

There is a substantial body of art describing various materials and techniques for using biocompatible implants in the human body. Some of the more important needs are for hip joints, knee and spine reconstruction and shoulder joints. These implants are usually metallic since they are load bearing structures and require relatively high strengths. To insure proper fixation to the bone, a porous metal coating is applied to the implant surface, and is positioned such that it is in contact to the bone. This is to promote bone growth into and through the porous coating to insure a strong bond between the bone and the implant. This coating requires a high degree of porosity, typically greater than 50% and as high as 70-80% with open connective pore sizes varying from 100μ to 500μ. These implants must have significant compression strength to resist loads that these joints can experience. The modulus must also be closely matched to that of the bone to avoid stress degradation of the adjoining tissue.

In addition to the use of tantalum fibers for bone growth, it can also be used effectively as a scaffold for soft tissue growth and can provide either a permanent or temporary support to the damage tissue/organ until functionalities are restored. Regardless of whether it's soft or hard tissue repair/replacement, all biomaterial will exhibit specific interactions with cells that will lead to stereotyped responses. The ideal choice for any particular material and morphology will depend on various factors, such are osteoinduction, Osteoconduction, angiogenesis, growth rates of cells and degradation rate of the material in case of temporary scaffolds.

Tissue engineering is a multidisciplinary subject combining the principles of engineering, biology and chemistry to restore the functionality of damaged tissue/organ through repair or regeneration. The material used in tissue engineering or as a tissue scaffold can either be naturally derived or synthetic. Further classification can be made based on the nature of application such as permanent or temporary. A temporary structure is expected to provide the necessary support and assist in cell/tissue growth until the tissue/cell regains original shape and strength. These types of scaffolds are useful especially in case of young patients where the growth rate of tissues are higher and the use of an artificial organ to store functionality is not desired. However, in the case of older patients, temporary scaffolds fail to meet the requirements in most cases. These include poor mechanical strength, mismatch between the growth rate of tissues and the degradation rate of said scaffold. Thus older patients need to have a stronger scaffold, which can either be permanent or have a very low degradation rate. Most of the work on scaffolding has been done on temporary scaffolds owing to the immediate advantages realized of the materials used and the ease of processing. Despite early success, tissue engineers have faced challenges in repairing/replacing tissues that serve predominantly biomechanical roles in the body. In fact, the properties of these tissues are critical to their proper function in vivo. In order for tissue engineers to effectively replace these load-bearing structures, they must address a number of significant questions on the interactions of engineered constructs with mechanical forces both in vivo and in vitro.

Once implanted in the body, engineered constructs of cells and matrices will be subjected to a complex biomechanical environment, consisting of time-varying changes in stresses, strains, fluid pressure, fluid flow and cellular deformation behavior. It is now well accepted that these various physical factors have the capability to influence the biological activity of normal tissues and therefore may plays an important role in the success or failure of engineered grafts. In this regard, it is important to characterize the diverse array of physical signals that engineered cells experience in vivo as well as their biological response to such potential stimuli. This information may provide an insight into the long-term capabilities of engineered constructs to maintain the proper cellular phenotype.

Significant advances have been made over the last four decades in the use of artificial bone implants. Various materials ranging of metallic, ceramic and polymeric materials have been used in artificial implants especially in the field of orthopedics. Stainless steel (surgical grade) was widely used in orthopedics and dentistry applications owing to its corrosion resistance. However later developments included the use of Co—Cr and Ti alloys owing to biocompatibilities issues and bio inertness. Currently Ti alloys and Co—Cr alloys are the most widely used in joint prostheses and other biomedical applications such as dentistry and cardio-vascular applications. Despite the advantages of materials such as Ti and Co—Cr and their alloys in terms of biocompatibility and bio inertness, reports indicated failure due to wear and wear assisted corrosion. Ceramics was a good alternative to metallic implants but they too had their limitation in their usage. One of the biggest disadvantages of using metals and ceramics in implants was the difference in modulus compared to the natural bone. (The modulus of articular cartilage varies from 0.001-0.1 GPa while that of hard bone varies from 7-30 GPa). Typical modulus values of most of the ceramic and metallic implants used lies above 70 GPa. This results in stress shielding effect on bones and tissues which otherwise is useful in keeping the tissue/bone functional. Moreover rejection by the host tissue especially when toxic ions (in the alloy such as Vanadium in Ti alloy) are eluted causes discomfort in patients necessitating revisional operations to be performed. Polymers have modulus within the range of 0.001-0.1 GPa and have been used in medicine for applications which range from artificial implants, i.e., acetabular cup, to drug delivery systems owing to the advantages of being chemically inert, biodegradability and possessing properties, which lies close to the cartilage properties. With the developments in the use of artificial implants there were growing concerns on the biocompatibility of the materials used for artificial implants and the immuno-rejection by the host cells. This led to the research on the repair and regeneration of damaged organs and tissues, which started in 1980 with use of autologuous (use of grafts from same species) skin grafts. Thereafter the field of tissue engineering has seen rapid developments from the use of synthetic materials to naturally derived material that includes use of autografts, allografts and xenografts for repair or regeneration of tissues.

Surface terrain or topography is one of the important factors governing cell adhesion and proliferation, and there have been many studies carried out in recent times to investigate the suitability of materials such as spider webs and cover slips, fish scales, plasma clots, and glass fibers. Silk fibers also have been used extensively in surgical applications such as for sutures and artificial blood vessels. Cell adhesion to materials is mediated by cell-surface receptors, interacting with cell adhesion proteins bound to the material surface. In aiming to promote receptor medicated cell adhesion the polymer surface should mimic the extracellular matrix (ECM). ECM proteins, which are known to have the capacity to regulate such cell behaviors as adhesion, spreading, growth, and migration, have been studied extensively to enhance cell-material interactions for both in vivo and in vitro applications. However, the effects observed for a given protein have been found to vary substantially depending on the nature of the underlying substrate and the method of immobilization. In biomaterial research there is a strong interest in new materials, which combine the required mechanical properties with improved biocompatibility for bone implants and soft tissue repair and replacement.

The foregoing discussion of the prior art derives from an article by Yarlagadda, et al. entitled Recent Advances and Current Developments in Tissue Scaffolding, published in Bio-Medical Materials and Engineering 15(3), pp. 159-177 (2005).

See also U.S. Pat. No. 5,030,233 to Ducheyne, who discloses a mesh sheet material for surgical implant formed of metal fibers having a fiber length of about 2 mm to 50 mm, and having a fiber diameter of about 20 to about 200 μm. According to the '233 patent if the fiber length is more than about 50 mm, the manufacturing becomes difficult. In particular, for fiber lengths in excess of about 50 mm, sieving the fibers becomes impractical if not impossible. If the diameter of the fibers is less than about 20 microns, it is difficult to maintain the average pore size of at least 150 μm needed to assure ingrowth of bony tissue. If the fiber diameter is greater than about 200 μm, the flexibility and deformability become insufficient.

As can be seen from the above discussion of the prior art, most of the research to date has been directed towards man-made implant material for rigid structures. Accordingly, there also exists a need for man-made implant material for promoting soft tissue growth as well.

### SUMMARY OF THE INVENTION

In my co-pending U.S. provisional application Ser. No. 61/266,911, and U.S. provisional application Ser. No. 61/295,063, incorporated herein, in their entireties, by reference, I disclose the use of valve metal fibers, such as tantalum for forming porous coatings on implants. In my co-pending U.S. provisional application Ser. No. 61/314,878, incorporated herein, in its entirety, by reference, I have now found that such valve metal fibers advantageously may also directly be used as a scaffolding for promoting soft tissue growth such as for nerves, tendons and cartilage, and also including other body parts. Such materials can also be used for sutures.

### BRIEF DESCRIPTION OF THE DRAWINGS

Further features and advantages of the present invention will be seen from the following detailed description taken in conjunction with the accompanying drawings, wherein:

FIG. 1 is a schematic block diagram of one alternative process of the present invention;

FIG. 2 is a simplified side elevational view showing casting of a sheet in accordance with the present invention; and

FIG. 3 is a side elevational view of a scaffolding implant in accordance with the present invention; and

FIGS. 4 and 5 are schematic block diagrams, similar to FIG. 1 of alternative processes of the present invention.

### DETAILED DESCRIPTION

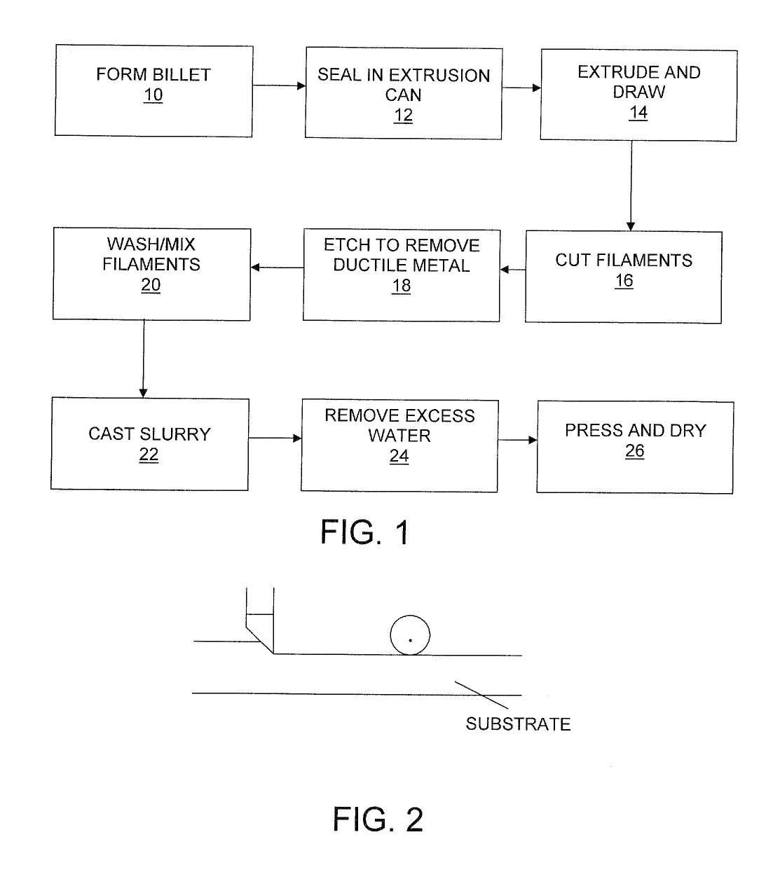

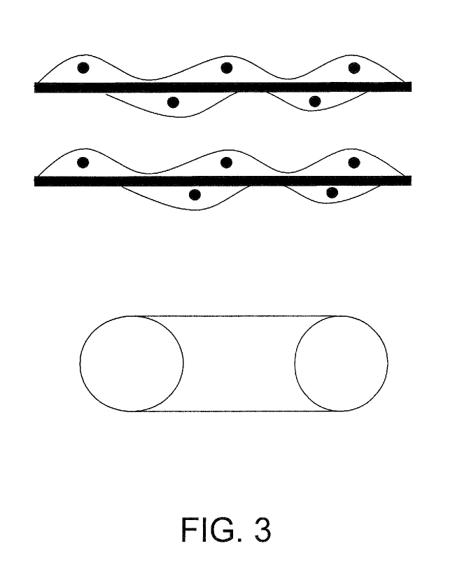

Referring to FIGS. 1 and 2, the process starts with the fabrication of valve metal filaments, such as tantalum, by combining shaped elements of tantalum with a ductile material, such as copper to form a billet at step 10. The billet is then sealed in an extrusion can in step 12, and extruded and drawn in step 14 following the teachings of my prior PCT applications Nos. PCT/US07/79249 and PCT/US08/86460, or my prior U.S. Pat. Nos. 7,480,978 and 7,146,709. The extruded and drawn filaments are then cut or chopped into short segments, typically 1/16^{th}-¼^{th} inch long at a chopping station 16. Preferably the cut filaments all have approximately the same length. Actually, the more uniform the filament, the better. The chopped filaments are then passed to an etching station 18 where the ductile metal is leached away using a suitable acid. For example, where copper is the ductile metal, the etchant may comprise nitric acid.

Etching in acid removes the copper from between the tantalum filaments. After etching, one is left with a plurality of short filaments of tantalum. The tantalum filaments are then washed in water in a washing station 20, and the wash water is partially decanted to leave a slurry of tantalum filaments in water. The slurry of tantalum filaments in water is uniformly mixed and is then cast as a thin sheet using, for example, in FIG. 2 a “Doctor Blade” casting station 22. Excess water is removed, for example, by rolling at a rolling station 24. The resulting mat is then further compressed and dried at a drying station 26.

It was found that an aqueous slurry of chopped filaments will adhere together and was mechanically stable such that the fibers could easily be cast into a fibrous sheet, pressed and dried into a stable mat. Notwithstanding, as long as the filaments are not substantially thicker than about 10 microns, they easily adhere together. Filaments that are much larger than about 50 microns, do not to form a stable mat. Thus, it is preferred that the filaments have a thickness of less than about 20 microns, and preferably less than about 10 microns, and preferably below 1 micron thick. To ensure an even distribution of the filaments, and thus ensure production of a uniform mat, the slurry preferably is subjected to vigorous mixing by mechanical stirring and vibration. The porosity of the resulting tantalum fibrous sheet can be varied simply by pressing the mat further. Also, if desired, multiple layers may be stacked together to form thicker sheets.

The resulting fibrous mat or sheet 30 is flexible and has sufficient integrity so that it can be handled and shaped into an elongate scaffolding where it can then be used. The fibrous mat product made according to the present invention forms a porous surface of fibers having minimum spacings between fibers of approximately 100 to 500 microns which encourages healthy ingrowth of bone or soft tissue.

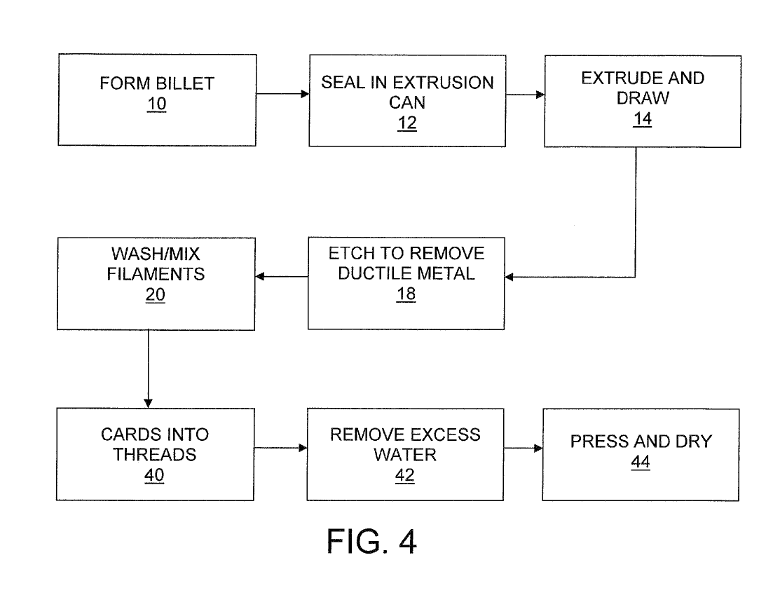

FIG. 3 illustrates the use of uncut continuous fibers, typically less than 20 μl in diameter, in a parallel orientation. Cells like those illustrated such as neurons can now adhere to the fiber surface and thus provide a scaffolding for synapse to connect and grow. Similarly for sutures, these long length fibers can simple be made by twisted together multiple fibers.

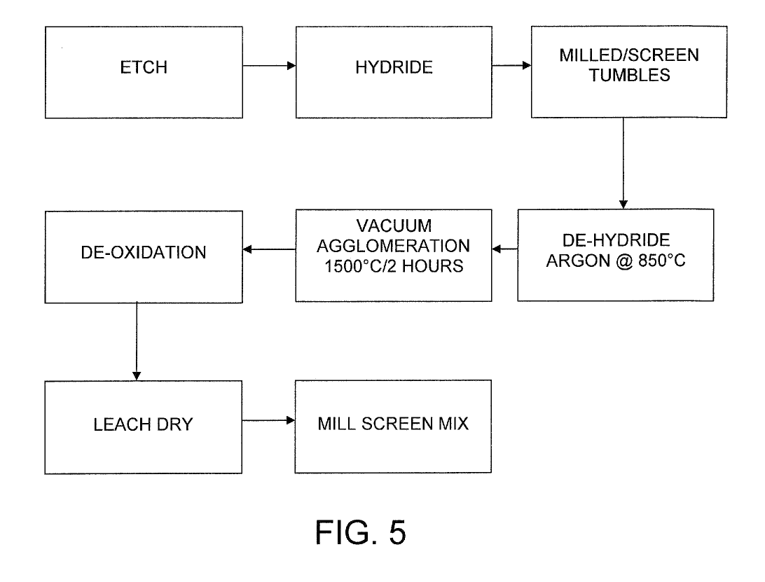

Numerous other arrangement by carding the fibers, meshes, braids and other fabric type arrangement can also be constructed as shown in FIG. 4.

Titanium powders for medical implants are often prepared using a hydride dehydride process (HDH). The powders are often irregular and angular in shape. When required these powders are often agglomerated to form larger particle by means of high temp vacuum sintering. In another embodiment of this invention shown in FIG. 5, the long Ta fibers are hydrided, crushed, dehydrided and agglomerated in similar fashion. This fiber-powder can now be used in exactly the same manner as solid metal powders are today. This process avoids the difficulties inherent with solid metals powders, and combines with it the advantages of using fibers. Higher porosity structures are now attainable by nature of the open pore structure which now consists of a bimodal network structure of interconnected open pores.

The present invention provides significant advantages over the prior art. For one, the fibrous product is extremely flexible, an important consideration where soft tissue growth is desired. Applicant's invention permits formation of fibrous elements significantly smaller than reasonably possible by conventional metallurgical techniques, and eliminates problems of potential contamination that result from conventional wire drawing techniques. Moreover, Applicant is able to form multi-filaments of various shapes and diameters including ribbons which are advantageously shaped. Applicant also is able to provide mats with filaments of different sizes and lengths which could further be advantageous in encouraging good fixation of tissue ingrowth.

While the invention has been described in connection with the formation of sheets or mats of tantalum fibers, other valve metals, such as titanium, zirconium, niobium or an alloy of two or more of said metals may be formed. Also, if desired, the metal fibers may be anodized making them electrochemically non-conductive. Still other changes may be made without departing from the spirit and scope of the invention.

1. A tissue implant member for implanting in living tissue, comprising a fibrous mat of valve metal filaments in which the filaments have a thickness of less than about 20 microns.

2. The implant as in claim 1, wherein said valve metal filaments have a thickness of 0.5 to 10 microns.

3. The implant of claim 1, wherein said metal filaments comprise niobium, titanium, tantalum or zirconium filaments.

4. The implant of claim 1, wherein said metal filaments comprise alloys of two or more metals selected from the group consisting of niobium, tantalum, titanium and zirconium.

5. The implant of claim 1, wherein the filaments are anodized.

6. The implant of claim 1, wherein the implant comprises a tissue scaffold for supporting soft tissue growth.

7. The implant of claim 6, wherein the tissue is selected from the group consisting of bone, nerve cells, tendons, cartilage and body organ parts.

8. A method for promoting soft tissue growth in a body comprising implanting in the body a tissue implant member as claimed in claim 1.

9. The method of claim 8, wherein the tissue is selected from the group consisting of bone, nerve cells, tendon or cartilage and body organ parts.

10. A tissue implant member for implanting in living tissue of animals, comprising elongate threads or yarn of valve metal filaments in which the filaments have a thickness of less than about 20 microns.

11. The implant as in claim 10, wherein said valve metal filaments have a thickness of 0.5 to 10 microns.

12. The implant of claim 10, wherein said metal filaments are selected from the group consisting of niobium, titanium, tantalum and zirconium filaments.

13. The implant of claim 10, wherein said metal filaments comprise alloys of two or more metals selected from the group consisting of niobium, tantalum, titanium and zirconium.

14. The implant of claim 10, wherein the filaments are anodized.

15. The implant of claim 10, wherein the implant comprises a soft tissue scaffold for supporting tissue growth.

16. The implant of claim 15, wherein the tissue is selected from the group consisting of bone, nerve cells, tendon, cartilage and body organ parts.

17. The implant of claim 10, wherein the implant comprises a suture.

18. A method for promoting soft tissue growth in a body comprising implanting in the body a tissue implant member as claimed in claim 10.

19. The method of claim 18, wherein the tissue is selected from the group consisting of bone, nerve cells, tendon, cartilage and body organ parts.

20. Sutures made from the tissue implant member of claim 1.

21. The implant of claim 1, where in the filaments are hydrided, crushed, dehydrided and agglomerated.

22. Sutures made from the tissue implant member of claim 10.
